(19) United States
(12) Patent Application Publication
(10) Pub. No.: US 2002/0151851 A1
(43) Pub. Date: Oct. 17, 2002

(54) Safe needle cover

(76) Inventor: Tun-Kuo Fu, Tainan (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/835,332

(22) Filed: Apr. 17, 2001

(51) Int. Cl. A61M 5/32

(52) U.S. Cl. 604/192; 604/263; 206/364; 206/365

(57) ABSTRACT

A safe needle-head cover in this invention is provided with a properly elongated slot in a circumferential wall for transversely receiving the needle after used in order not to stab and hurt a user by accident. Besides, a manifest mark is printed on an edge of the slot to let the user easily catch sight of the exact position of the slot, and then the slot of the needle cover is sealed by a sealing tape after manufactured to permit the needle kept clean inside before using.

### BACKGROUND OF THE INVENTION

This invention relates to a safe needle cover, particularly to one possible to prevent a user from accidentally stabbed and hurt by a used needle when reinserting it in the needle cover together ready to be cast away.

It is commonly known that germs are varied and everywhere, and human bodies are easily infected by such germs or other virus, and liable to fall sick. In case of illness, a man had better go to see a doctor, taking medicine or having injection, and injection has become one of the most popular ways of treatment.

Conventionally, an injector, referring to FIGS. 6 and 7, is composed of a hollow barrel 2 having inside a plunger 4 connected with a piston 5, a needle 3 fixedly connected to the front end of the hollow barrel 2 and a needle cover 6 to receive the needle.

When being used, the needle 3 is pulled out of the needle cover 6, and, after used, reinserted into the needle cover 6, avoiding stabbing and hurting anyone else due to randomly discarding.

However, in the process of straightly reinserting the needle into the needle cover, a user is liable to be stabbed hurt by the needle due to carelessness, and even worse, to be infected by virus coming from the patient who is just injected.

### SUMMARY OF THE INVENTION

One objective of the invention is to offer a safe needle cover having an elongated slot provided on a circumferential wall for facilitating a needle to be reinserted transversely into the needle cover to ensure safety in using.

Another objective of the invention is to offer the safe needle cover, which is transparent and has an attractive mark on an edge of the slot for a user to easily catch sight of the exact position of the slot.

The third objective of the invention is to offer the safe needle cover with its slot sealed over by means of a sealing tape after manufactured to let the needle head kept clean inside, and this sealing tape is to be torn off when the needle head is to be used.

### BRIEF DESCRIPTION OF THE DRAWINGS

This invention will be better understood by referring to the accompanying drawings, wherein:

FIG. 1 is an exploded perspective view of a safe needle-head cover of the present invention.

FIG. 2 is a partial magnified exploded perspective view of the safe needle-head cover of the present invention.

FIG. 3 is a cross-sectional view of the safe needle-head cover of the present invention.

FIG. 4 is a magnified perspective view of a second embodiment of the safe needle-head cover with a manifest mark on an edge of a slot of the present invention.

FIG. 5 is a magnified perspective view of a third embodiment of the safe needle-head cover with its slot sealed over of the present invention.

FIG. 6 is a perspective view of a known conventional needle-head cover.

FIG. 7 is a cross-sectional view of the known conventional needle-head cover.

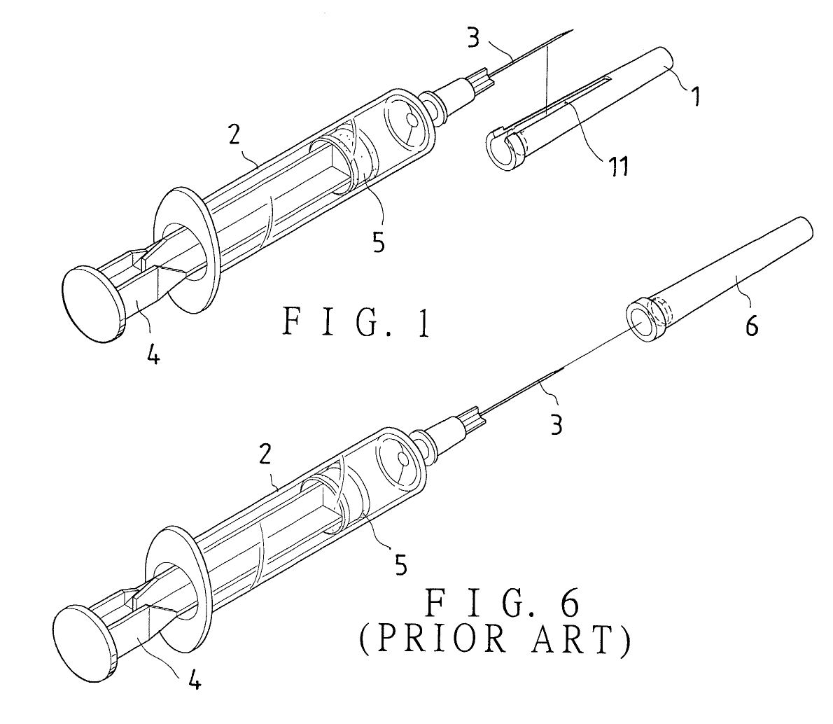

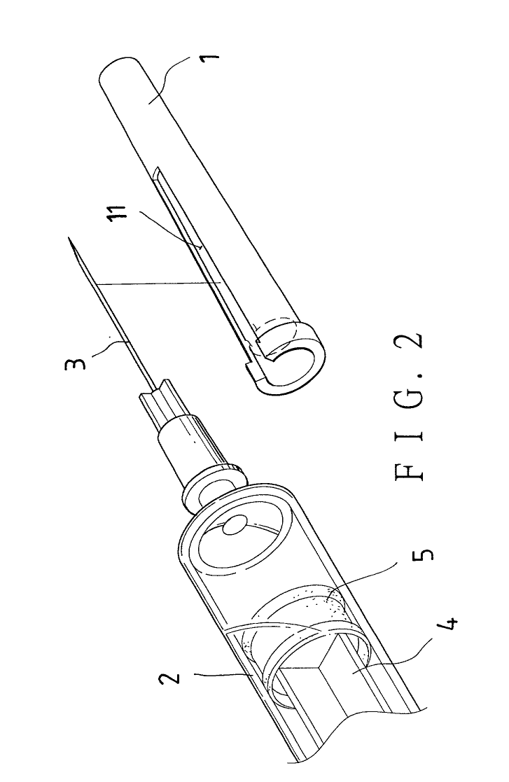

### DETAILED DESCRIPTION OF THE PREFERRED EMBODIMENTS

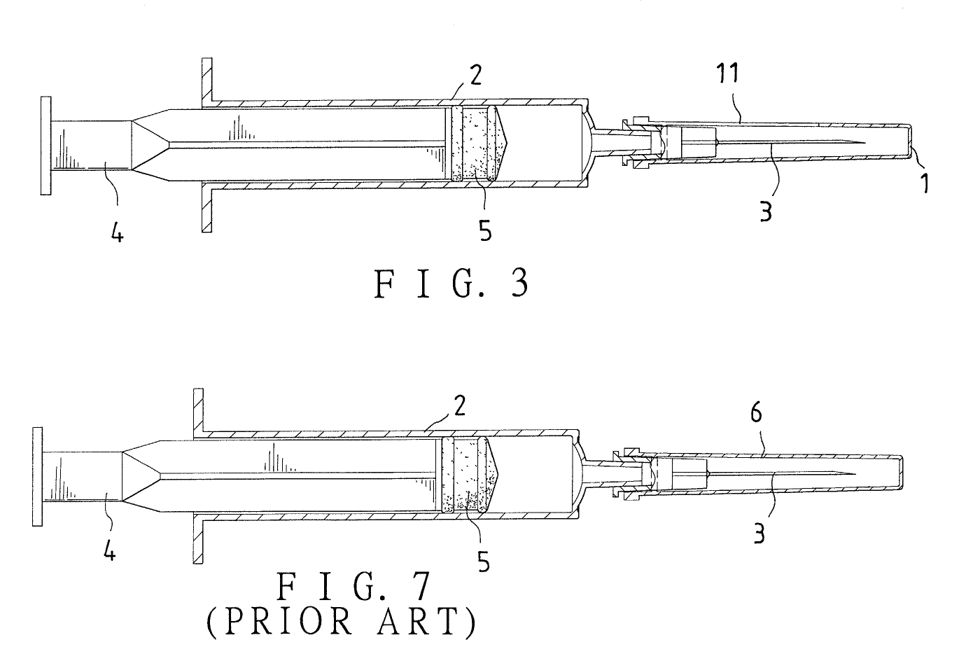

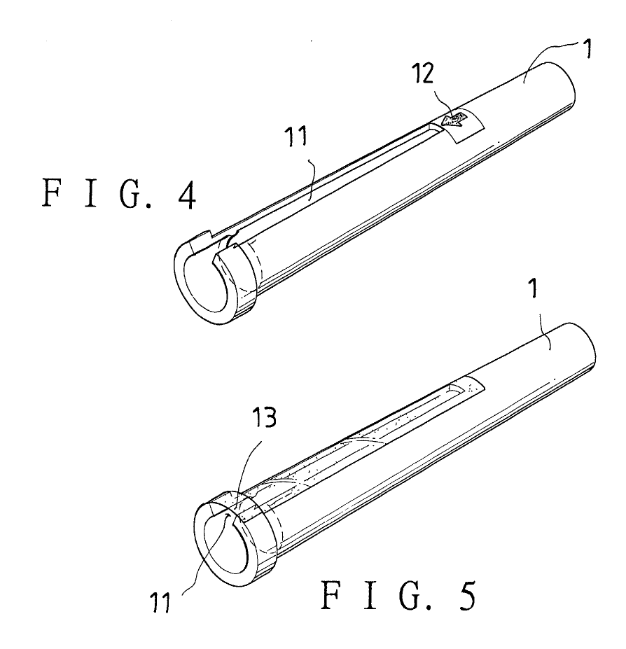

A preferred embodiment of a safe needle-head cover 1 in the present invention, as shown in FIGS. 3 and 4, is provided conforming to a known conventional needle 3 fixedly connected to a hollow barrel 2 which has inside a piston 5 connected with a plunger 4.

The safe needle cover has a properly elongated slot 11 formed at a proper position in a circumferential wall for exactly receiving the needle 3.

In using, referring to FIG. 3, the needle, after finishing injection, is transversely placed into the slot 11 of the needle cover 1 which is to be fixed around the needle.

Further, referring to FIG. 4, the transparent needle-head cover 1 can be provided with a certain attractive mark on an edge of the slot 11 for a user to catch sight of the exact position of the slot immediately.

Furthermore, referring to FIG. 5, the slot 11 of the needle cover 1, after manufactured, is sealed over by means of a sealing tape 13 to let the needle kept clean inside, and, when to be used, just tear off the sealing tape 13.

As can be understood from the above description, this invention compared with the known conventional one, has the following advantages.

1. The safe needle-head cover is provided with a properly elongated slot in a circumferential wall to let the needle, after used, reinserted transversely into the needle cover, possible to prevent a user from stabbed and hurt by the needle in carelessly positioning it into the needle cover.

2. The safe needle-head cover is provided with a manifest mark on an edge of the slot so as to let a user immediately catch sight of the exact position of the slot.

3. The safe needle-head cover, after manufactured, has its slot sealed by means of a sealing tape to keep the needle head clean inside, and when to be used, just tear off the sealing tape.

What is claimed is:

1. A safe needle cover covering around a needle connected to a front end of a hollow barrel and a plunger movably fitted in said hollow barrel;
characterized by said needle cover having a properly elongated slot for transversely receiving said needle after used so as to prevent a user from stabbed and hurt by accident by said needle.

2. The safe needle cover as claimed in claim 1, wherein a certain attractive mark is printed on an edge of said slot of said needle cover to let a user immediately catch sight of the exact position of said slot.

3. The safe needle cover as claimed in claim 1, wherein said slot is sealed over by means of a sealing tape to let said needle unused kept clean inside said needle cover, and when to be used, said sealing tape is torn off.
